(19) United States
(12) Patent Application Publication
(10) Pub. No.: US 2006/0098825 A1
(43) Pub. Date: May 11, 2006

(54) Electronic adaption of acoustical stethoscope

(76) Inventor: Hart Victor Katz, Toronto (CA)

Correspondence Address: DENNISON ASSOCIATES, 133 RICHMOND STREET WEST, SUITE 301, TORONTO, ON, M5H 2L7, CA

(21) Appl. No.: 10/981,562

(22) Filed: Nov. 5, 2004

(51) Int. Cl. A61B 7/04; A61B 7/02; A61B 5/02

(52) U.S. Cl. 381/67; 600/528; 181/131

(57) ABSTRACT

A stethoscope adapter receives and converts a stethoscope transducer signal using a predetermined inverse function. The adapter then transmits the converted signal as an acoustical signal through a tube connector. With this arrangement, the adapter converts the signal to compensate for undesirable non linear transmission effects through known stethoscopes. The user continues to receive a signal of conventional characteristics and the signal is easily interpreted. Preferably, the adapter interacts with a PDA device to export wirelessly and in real time the signal for additional analysis. The PDA device preferably interacts with the adapter for programming thereof for a particular stethoscope and/or the operation thereof including the on/off function.

### FIELD OF THE INVENTION

The present invention relates to electronic stethoscopes and in particular to an adapter which inserts into a conventional stethoscope and amplifies and processes the signal in a manner to improve the outputted signal.

### BACKGROUND OF THE INVENTION

Conventional acoustical stethoscopes continue to be widely used by the medical practitioner as a useful preliminary screening and diagnostic tool. In particular, the stethoscopes are commonly used to investigate the mechanical function of the heart, lung and cardiovascular system. Medical practitioners compare the particular sounds being transmitted with acoustic templates they have learned and enhanced by their actual experience. The particular signals of interest are typically below 500 Hz.

Conventional acoustical stethoscopes use a mechanical transducer to detect the signals of interest. In addition, these stethoscopes also transmit background noise. The sound from the mechanical transducer is transmitted through a flexible tube to a bifurcated earset. The combined flexible tube and bifurcated earset subject both the narrow band signal, and broadband noise to the acoustic resonances of the tube.

In the conventional stethoscope, the intensity of background noise is low, and the application of the tube resonances to this noise is tolerable. This application becomes a significant problem, however if the signal is electronically amplified prior to transmission.

Prior art electronic adapters for conventional stethoscopes such as disclosed in my earlier U.S. Pat. No. 4,528,629, have amplified and filtered the signal prior to transmission but no filter to suppress the background noise component unfortunately has ever been designed or applied.

The human ear is very sensitive to wideband noise below 2 KHz and any sound with spectral intensities spread relatively evenly across this band becomes very noticeable when amplified and thus interferes with the interpretation of the heart and lung sounds of interest to the physician.

Although this background noise is present in mechanical stethoscopes, the noise is low in intensity and can be ignored. Some electronic stethoscopes provide an electrical transmission path between an electrical transducer and the output to the medical practitioner, however, the outputted sound is difficult to compare with the learned acoustic templates of the practitioner. Furthermore, this approach using the new electronic stethoscopes is expensive and requires significant investment in time to become familiar with the new device. Some of companies have packaged the product to appear similar to a conventional stethoscope to increase initial acceptance.

Other electronic stethoscopes provide at least a partial acoustic transmission path with selective filtering of the signal prior to acoustic transmission. Unfortunately, the desired amplification of the signal containing both the desired narrow band signal and wide band background noise renders the signal difficult to interpret.

For these and other reasons, electronic stethoscopes have not been widely accepted. There continues to be many models of conventional stethoscopes which remain a critical aid for most medical practitioners.

Suppression of background sound follows from the fact that it occupies a wider frequency bandwidth than the narrow band signal desired to be heard. Although both these broad and narrow band signals are amplified equally, thereby preserving the actual Signal to Noise Ratio (SNR), the ear and auditory cortex are more sensitive to the wideband background noise for two reasons:

1. Practically speaking, wideband noise being electronically amplified can be considered as white noise. However, after passage through the transmission tube, this noise is shaped, or colored by the resonances of the tube. These harmonics often clash with the net effect that the noise now is acoustically jarring and hence more noticeable; and.

2. The acoustic response to increasing sound pressure levels is non-linear beyond a certain level of sound pressure. Beyond this level, the response increases at a rate far greater than the below threshold linear rate. This phenomenon is known as recruitment and has been studied extensively.

For both these reasons, the noise level appears to increase disproportionately to the signal, and thus the perceived SNR drops considerably.

Thus, any filter designed to lower the intensity of the background noise to those levels sensed while using a mechanical stethoscope will increase the perceived SNR. In this way, the sound produced by the electronic device presents more like a mechanical device, and thereby can be interpreted by more conventional means.

### SUMMARY OF THE PRESENT INVENTION

The present invention recognizes that the stethoscope remains an important component of the physical exam as performed by the medical practitioner and this important component can be improved by an adapter that amplifies the signal and processes the signal to compensate for the effects of the transmission path of the tube prior to the acoustical transmission of the signal through the tube. Preferably, this processing embodies the application of a transfer function which approximates the inverse of the transfer function of the stethoscope tube itself. The inverse transfer function is preferably determined by investigating conventional accepted stethoscopes and constructing an acoustic model of the binaural tubes. Inverse transfer functions for each respective binaural tube of the conventional accepted stethoscopes are then used by the adapter prior to acoustical transmission.

In a preferred aspect of the invention, it is recognized that the utility of the stethoscope can be enhanced in three distinct ways, namely

1. Sharpening the acoustics through amplification and filtering, as is currently provided by electronic stethoscopes, providing the perceived signal to noise ratio after such modifications can remain at least no less than that observed signal to noise ratio encountered with mechanical stethoscopes;

2. Providing a visual display to accompany the listening to the sound, wherein the user may follow an onscreen cursor which sweeps synchronously with the playback, and which said playback may occur both at full speed, and half speed without noticeable distortion in pitch or tonality; and

3. Using software designed to analyze the sound according to clinically verified algorithms in an efficient manner so as to expedite diagnosis by the practitioner during the physical examination of the patient.

With regard to the acoustics, the present invention seeks to retain the advantages and familiarity of the mechanical stethoscope by modifying it as little as possible. Specifically, the present invention provides for a battery operated adapter which fits into the stethoscope tube and leaves both the chest piece and binaural earpiece unchanged, thereby maintaining the quality of the stethoscope sound. The adapter provides the electronic amplification and conditioning of the sound digital processing of the adapter and includes the inverse transfer function to reduce the resonance distortion of the transmission tube.

This noise suppression technique is based on the fact that the apparent increase in noise arises from excitation of the mechanical resonances of the stethoscope binaural tube, and that the noise can be suppressed by modeling the binaural using a digital linear filter, and applying the inverse of this filter to the sound prior to introducing it to the binaural.

This noise suppression amplifies the signal while maintaining the acoustic quality of a mechanical stethoscope. The adapter also allows communication of the signal to related components for analysis, if desired.

With regard to simultaneous display and playback of the sound signal, the invention provides for the adapter to transmit wirelessly and in real time to a remote processor which can display and playback the signal immediately after having received it. In this fashion, the user can take advantage of both sight and sound to better interpret the sounds.

With regard the analysis of the sound on the remote processor, the invention also provides for a complete database of documented sounds which are available to the user for immediate comparison. Specific to this database, the invention offers a Windows Explorer type database which can be used to access any given sound according to the underlying physiological principles which are responsible for generating the sound. In this fashion, simply by using the Explorer tree effectively, the clinician can diagnose the pathology simultaneously with finding it in the database.

An adapter for a stethoscope, according to the present invention, comprises an input having a tube connector for audibly connecting with a tube leading to a stethoscope sensor, a battery operated processor for receiving an audio input through said tube connector and converting said audio input to a digital signal and modifying the digital signal to compensate for a predetermined stethoscope distortion and an acoustical output driven by said modified signal digital signal and a tube connector for connection with a stethoscope transmission tube arrangement.

According to another aspect of the invention, the adapter can be remotely adjusted with regard the application of signal gain and filtering, such that these adjustments will remain in place in the adapter until changed at a later time.

According to yet a further aspect of the invention, the adapter includes an output transmission function for transmission or the digital signal to a remote receiver.

Yet a further aspect of the invention, the output transmission function of the adapter transmits the digital signal in a wireless manner.

In yet a further aspect of the invention, the remote receiver is a small battery operated computer.

In a different aspect of the invention, the adapter periodically polls the environment for a compatible wireless receiver, and commences digital wireless transmission to the receiver immediately upon recognition of that receiver. Thereinafter a two way communication channel is established wherein both data and control information may be transmitted between the two stations.

### BRIEF DESCRIPTION OF THE DRAWINGS

The above as well as other advantages and features of the present invention will be described in greater detail according to the preferred embodiments of the present invention in which;

FIG. 1 is a perspective view of a conventional stethoscope with the adapter inserted in the transmission tube arrangement thereof;

FIG. 2 is a schematic diagram showing a conventional stethoscope with the adapter in IR transmission and communication with a PDA device;

FIG. 3 is a schematic diagram of signal processing of the adapter; and

FIG. 4 is a schematic diagram of a signal processed through a stethoscope having the signal adapter.

### DETAILED DESCRIPTION ACCORDING TO THE PREFERRED EMBODIMENTS OF THE PRESENT INVENTION IN WHICH

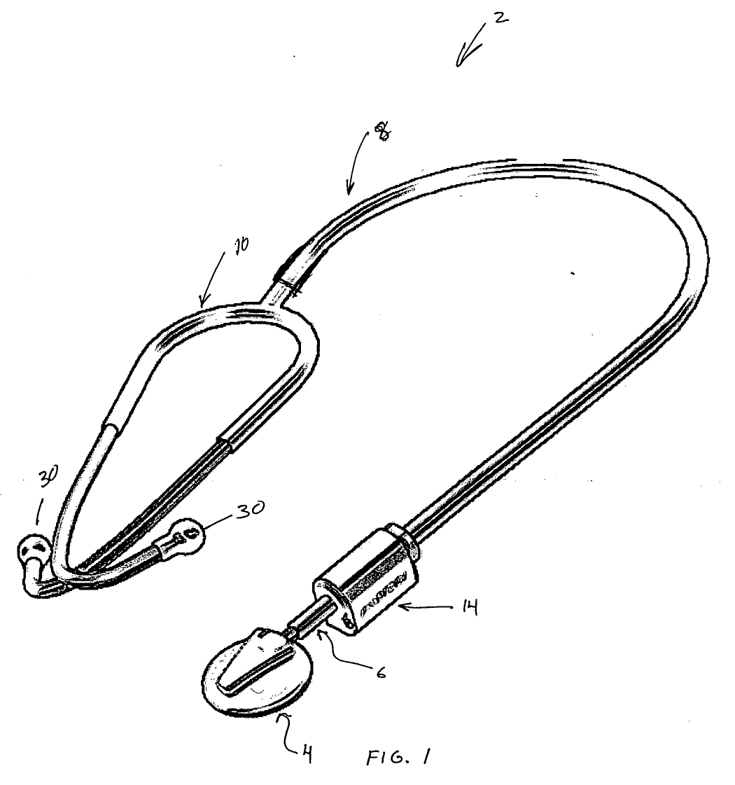

A conventional stethoscope 2 is shown in FIG. 1 where the adapter 14 has been inserted a short distance from the chestpiece 4 containing an acoustical transducer. The adapter 14 is connected to the chestpiece by short transition tube 6. The output of the adapter 6 is connected by the normal transmission tube 8 to the bifurcated earset 10. In this way, a conventional stethoscope has been adapted by means of inserting the adapter 14 between the chestpiece 4 and the tube transmission arrangement 8 and if desired the stethoscope can be returned its original configuration. Typically, the adapter 14 is provided with the short transmission tube 6 to allow connection to the chestpiece 4. Therefore the adapter can be removed and the transmission tube 8 reconnected to the chestpiece 4.

The modified stethoscope operates much in the manner of a conventional stethoscope however, the adapter 14 processes the acoustical signal received from the transducer 4 and provides a modified acoustical output signal to the transmission tube arrangement 6. The adapter 14 filters the signal, amplifies the signal, applies an inverse transfer function to the signal, and transmits a modified amplified acoustic signal to the transmission tube 8.

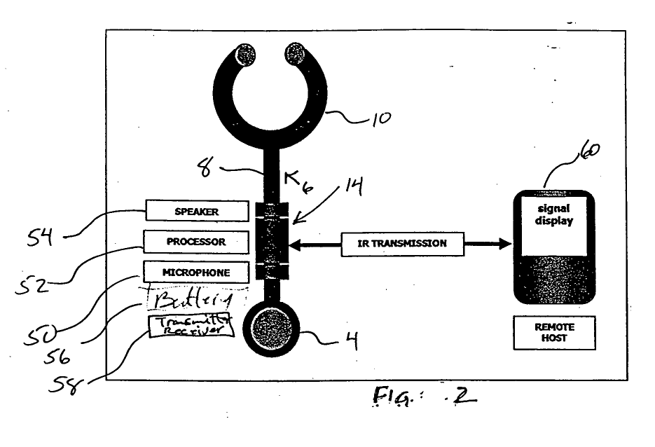

The adapter 14, as shown in FIG. 2, includes a microphone 50, a processor 52 and an output speaker 54. The adapter also includes a battery 56 and a transmitter/receiver 58.

Some separation of the adapter from the transducer is required to provide convenience of use in the normal manner of a conventional stethoscope and the advantageous positioning of the adapter for transmission to a remote device during use of the stethoscope. Preferably, the adapter 14, via the transmitter/receiver 58 communicates using IR (infrared) transmission to the PDA device 60.

This PDA device is particularly desirable as it is a portable device and commonly used by medical practitioners. PDA devices can be used to augment other diagnostic and therapeutic procedures, including presentation of databases for current drugs and their side effects, infectious diseases, and diagnostic trees. These same PDA devices can store programs to display, analyze, and store transmitted signals to enhance screening and diagnostic efficacy.

Although a separate PDA device is shown and is particularly desirable, it can be appreciated that the adapter could communicate with any remote computer which may be available to receive the wireless signal. Although the remote computer needs to be relatively close to allow a low power transmission, it need not be a portable device.

The chestpiece 4 of the traditional stethoscope senses internal body sounds that have propagated to the body surface, usually the chest and back. These sounds then propagate through the binaural tube to the ear outputs 30 of the bifurcated earset 10.

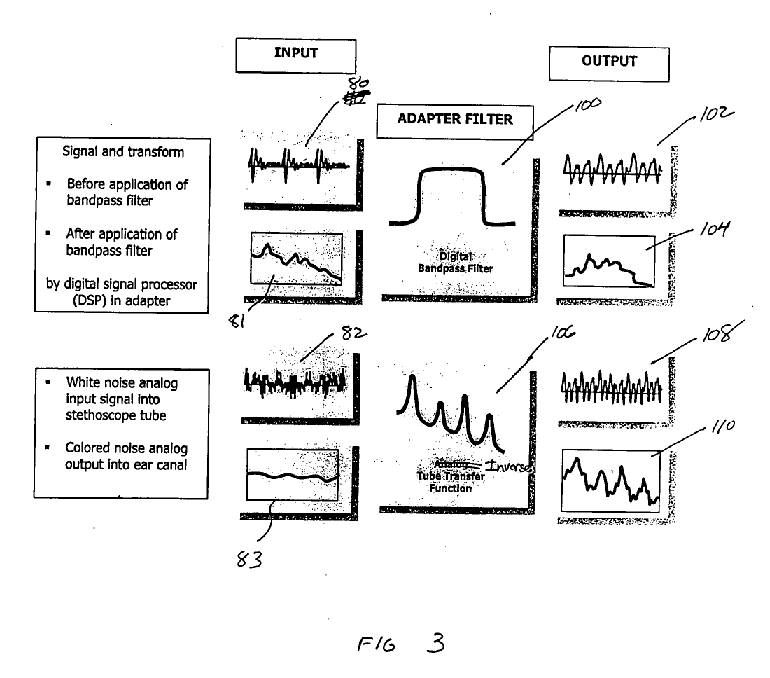

FIG. 3 provides an overview of the signals transmitted through the stethoscope.

The body sounds (heart, lung, etc.) acquired by the stethoscope are usually low frequency narrow band signals 80, while the background noise, arising both from the environment and interaction of the chest piece with the body surface, is a wideband signal similar to white noise indicated as 82. The narrow band signal 80 of the desired body sounds has a certain amplitude frequency distribution represented as 81, and the signal 82 has a certain amplitude frequency distribution 83. These sounds in a conventional stethoscope are modulated by the combination transmission tube 8 and bifurcated earset 10, which act as a cavity resonator in the sense that the combination amplifies both signal and noise at its resonance frequencies.

The wideband noise tends to excite most of these resonances, which can then be heard as a chord. Unfortunately, the resonance frequencies are not arranged in any harmonious relation, and the resulting sound is noticeable. In a conventional mechanical stethoscope, these discordant notes are soft, and can be easily, if not unconsciously, ignored by the user. In a case of the electronic stethoscope, however, both signal and noise are filtered and amplified by the adapter before presentation to the transmission tube 8 and the bifurcated earset.

Although both signal and noise are amplified in the same proportion, the higher intensity noise remains wideband and therefore triggers the resonant frequencies of the transmission tube 8 and the bifurcated earset 30. In addition, the noise is perceived as louder by virtue of the discordant notes it produces. In fact, the perception of this noise increases in a supra-linear fashion in a manner described in the neurological literature as “recruitment”. Basically, the user's ability to ignore the noise is less, and the desired amplification of the signal may actually reduce the ability to interpret the signal.

Thus, although the actual signal to noise ratio is unchanged by electronic amplification, the perceived signal to noise ratio diminishes considerably.

The adapter 8 of the present invention recognizes that the problem of electronic stethoscopes that amplify the signal and transmit an acoustic signal through a transmission tube and bifurcated earset, is the amplified noise component of the signal and the resonance of these components in the transmission path.

To overcome these problems, the adapter is designed to be used with one of a number of specific stethoscopes and the adapter applies a transfer function to the digital signal prior to acoustical transmission to the transmission tube and the bifurcated earset. The transfer function of the filter is the inverse of the transfer function of the particular transmission tube 8 and the earset 10.

Inverse transfer functions are predetermined for different models of conventional stethoscopes. Basically, the same model stethoscopes will have similar transfer functions and the adapter can be programmed using the inverse transfer function to eliminate or greatly reduce the problems associated with resonance.

In the present embodiment, the analog to digital converter converts the signal from an analog to a digital signal which is then conducted to the on-board processor and amplified, and filtered. The digital version of the inverse function is then applied to the digitized sound within the processor, immediately prior to conducting the digital signal to the digital to analog converter, and the resulting analog signal thereby directed to the speaker 54.

The speaker 54 then presents this signal to the transmission tube arrangement 8 and bifurcated earset 10. The transmission tube arrangement 8 and the bifurcated earset 10 modify the signal according to their generally known modulation characteristics. The resulting signal which presents at the ear outputs 30 again has been modified by the combined transmission arrangement 8 and the bifurcated earset 30. However, the undesirable resonance effects of the broadband noise has been reduced or eliminated due to the preprocessing of the signal to account for the modifications signal by the transmission arrangement.

In a preferred embodiment the PDA device 60 is used to program the adapter 14. Certain software for providing an instruction set to the adapter 14 is provided with the adapter and is loaded into the PDA device or other computer. The adapter processor is then programmed for the particular type of stethoscope in which it is to be inserted. As previously stated there are twenty or more commonly used stethoscopes and each of these stethoscopes are previously analyzed to determine their own inverse functions which are then all loaded into the software.

The user then merely selects the particular stethoscope and the appropriate inverse function is loaded into the adapter. In this way, simple programming of the adapter is provided.

The PDA device 60 is also used to activate the transmission link. The adapter routinely broadcasts an IR pulse and waits for a return pulse from any appropriate IR receiver, such as a PDA device which has been programmed to recognize the adapter.

Once the adapter senses a return pulse from a receiver, it starts to transmit to that receiver 58 embedded within the PDA device 60. In this way, the adapter remains in a very simple form and does not require various activation switches. The adapter includes memory available to the processor for the temporary storing of the transducer signal.

The battery supply 50 is typically a lithium battery supply and has the ability to last several months or more without battery replacement. In one embodiment, the user presses the on/off button located on the adapter to begin operation. In a second embodiment, the processor remains in sleep mode when not in use; in this mode it can sense a significant change in the input signal level and thereby wakeup to full operating mode.

For example, the medical practitioner might blow on the transducer or produce a signal a certain magnitude which activates the processor.

The operating parameters of the adapter can be modified remotely by the PDA. Specifically, the user may separately control the volume and the selection of various digital filters. These filters include the inverse transmission functions that suppress background noise, as discussed herein, and various band pass filters. With regard the filters, the user may select the inverse transmission function and one or none of several bandpass filters

The choice of the filter is determined by the pathology that the practitioner is seeking to assess. Furthermore, for transmitting to the PDA a signal to be recorded or analyzed, it may not be desirable to use the inverse function, or at least to transmit to the PDA, a signal that has not been modified using the inverse function.

To program the adapter, the user simply places the adapter within IR range of the PDA, and uses a program to select the desired parameters which are then transmitted back to the adapter. These settings remain in place until changed.

Furthermore, the PDA can be programmed to display, analyze, playback the sounds through the onboard soundcard, or transmit the sounds to a remote computer platform for permanent archiving. In this regard, the PDA can be used for increasingly sophisticated analysis and over time, the knowledge base of the medical practitioner will continue to increase relative to his existing knowledge of particular conditions sensed by a stethoscope.

FIG. 3 is a simplification of the processing by the adapter 14 and the output signal broken into the two major components. A bandpass filter 100 restricts the signal to the desired frequency range that sill includes the desired signal and noise. The output signal 102 is generally produced, based on signal 80 and the amplitude frequency distribution as shown at 104 is similar to the bandpass filter 100 as expected.

The noise signal 82 is reduced by the bandpass filter and is also modified by the inverse tube transfer function 106. Due to the wideband nature of the noise, it has frequencies which excite resonance, and the frequencies are modified by the inverse transfer function. The signal 108 is simplified relative to input 82, and the frequency amplitude distribution 110 is similar to the inverse function.

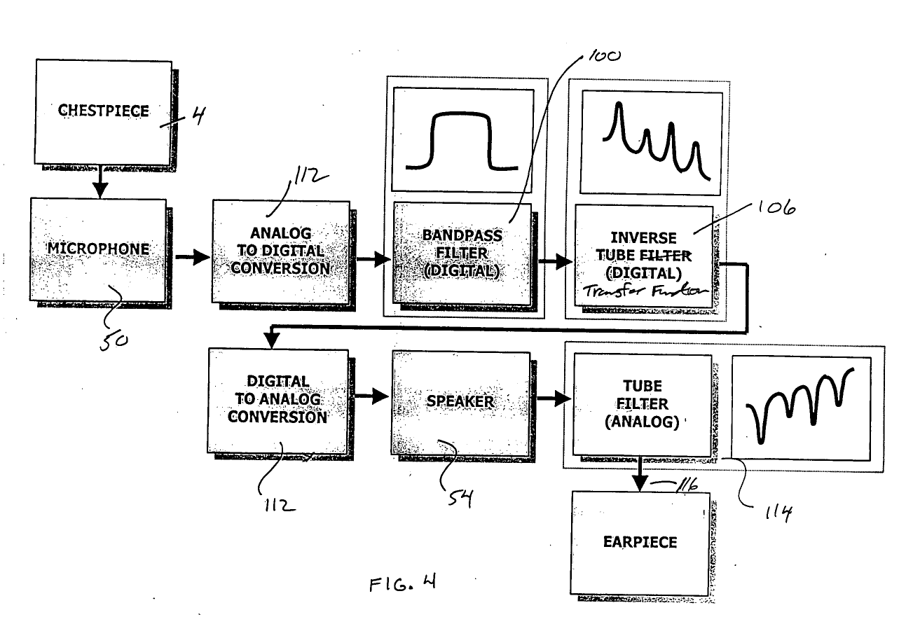

The results of the combined adapter and transmission tube, and earset of a conventional stethoscope, are illustrated in FIG. 4.

The adapter 14 includes the microphone 50 to receive the signal from the chestpiece 4. The received signal is converted from analog to digital at 112. Amplification occurs in the adapter and the bandpass filter 100, and the inverse tube transfer function 106 is applied to the signal. The digital signal is converted to analog at 112 and provided to the speaker 54. The combined transmission tube function and earset modify the signal at 114, and an output signal is provided at 116. The inverse transfer function and the combined transmission tube function both act as multipliers and therefore, the signal is not substantially changed. Thus, the undesirable effects of resonance in the transmission tube and earset are reduced.

The position of insertion of the adapter has been described relative to the end of the stethoscope tube and the inverse function for this position is provided. As can be appreciated there may be applications where a different position of the adapter is desired and inverse functions for different positions can be provided.

Although various preferred embodiments of the present invention have been described in detail, it will be appreciated by those skilled in the art that variations may be made without departing from the spirit of the invention or the scope of the appended claims.

The embodiments of the invention in which an exclusive property or privilege is claimed are defined as follows:

1. An adapter for a stethoscope comprising an input having a tube connector for audibly connecting with a tube leading to a stethoscope sensor, a battery operated processor for receiving an audio input through said tube connector and converting said audio input to a digital signal, modifying said digital signal to compensate for a predetermined stethoscope distortion that occurs during the transmission of an output signal from said adapter, and an audio output connectable to earpieces of a stethoscope.

2. An adapter as claimed in claim 1 wherein said adapter is selectively operable to apply selected gains and filters to enhance certain frequencies within the digital signal for the purposes of diagnoses, or to suppress the effects of unwanted modulation of the signal by the stethoscope tube.

3. An adapter as claimed in claim 1 including an output transmission function for transmission of said digital signal to a remote receiver.

4. An adapter as claimed in claim 3 wherein said output transmission function transmits said digital signal in a wireless manner.

5. An adapter as claimed in claim 4 wherein said remote receiver is a small battery operated computer.

6. An adapter as claimed in claim 1 wherein said output transmission function includes a transmitter and receiver and said transmitter periodically broadcasts a pulse coded sequence in search of a compatible wireless receiver, and upon receipt of a response, initiates transmission to said remote receiver.

7. An adapter as claimed in claim 6 wherein said adapter includes a series of programming options for processing of said digital signal and programming of said adapter is carried out by sending instruction signals to said adapter from a remote computer in two way communication with said adapter via said transmission output function.
